SEQUENCE LISTING (2 sequences)

SEQ ID NO: 1 - protein, 10 aa, Aedes aegypti
NTGDKFYGLM

SEQ ID NO: 2 - protein, 10 aa, Aedes aegypti
DTGDKFYGLM

(19) United States
(12) Patent Application Publication
(10) Pub. No.: US 2003/0175286 A1
(43) Pub. Date: Sep. 18, 2003

(54) Use of mosquito salivary tachykinins to remediate unregulated cellular proliferation

(76) Inventors: Nathan Maier, Longview, TX (US); Amiel G. Jarstfer, Longview, TX (US)

Correspondence Address: John P. O'Banion, O'BANION & RITCHEY LLP, Suite 1550, 400 Capitol Mall, Sacramento, CA, 95814, US

(21) Appl. No.: 10/267,918

(22) Filed: Oct. 8, 2002

Related U.S. Application Data
(63) Continuation of application No. 09/713,299, filed on Nov. 16, 2000, now abandoned.

(51) Int. Cl. A61K 38/43; A61K 39/00; C07K 14/435

(52) U.S. Cl. 424/185.1; 530/395

(57) ABSTRACT

Administration of mosquito salivary tachykinins, such as sialokinin I and II, topically or by injection to an area of unregulated cellular proliferation will result in the recovery of cellular proliferative control. The tachykinins cause a number of physiological and immune system changes which induce recovery of proliferative control. These changes include vasodilation, increased vascular permeability, activation of macrophages, activation of neutrophil granulocytes, T-lymphocyte proliferation, monocyte interleukin production, mast cell degranulation in epithelia and eosinophyles; all of which function to stimulate the body to regain the delicate proliferative balance which has been disturbed by various environmental and viral insults on the body such as UV radiation, ionizing radiation, HPV, and other insults that cause tumors or warts through the same mechanisms.

### BACKGROUND OF THE INVENTION

1. Field of the Invention

The present invention involves the treatment of unregulated areas of cellular proliferation “usually referred to as tumors or warts” in the mammalian body by administering specific tachykinins to those areas of unregulated cellular proliferation.

2. Discussion of the Prior Art

Human papilloma virus (HPV) infection is an example of a viral disruption of the proliferative balance in cell cycle control. It typically begins by inoculation of an epithelial wound with the viral particles (Ragland et al., 1994). The HPV's double stranded DNA is taken in by a nearby cell and commandeers the host cell's transcriptional machinery to coordinate the expression of viral gene products in a specific spatiotemporal sequence within the differentiating epithelial layer. In this way the early genes are expressed in proliferating undifferentiated keratinocytes (Saunders and Frazer, 1998). The HPV proteins E5, E6, and E7 (See FIG. 1.), are responsible for HPV-associated tumor development. The E5 protein inactivates tumor suppressor gene p21 and stimulates human growth factor activity, enhancing cell proliferation and possibly influencing transformation to malignancy (Flaitz and Hicks, 1998; Saunders and Frazer, 1998). Protein E6 binds to the tumor suppressor p53 gene targeting it for destruction (Saunders and Frazer, 1998), as well as binding to the p53 protein which has been referred to as the ‘guardian of the genome’ (Flaitz and Hicks, 1998). When activated by mutational stimuli, such as ultraviolet- or gamma-irradiation, p53 induces the expression of gene product p21, which inhibits the cell cycle until such time as any DNA damage is repaired. If the DNA damage is too great, then p53 induces apoptosis. Thus, the inactivation of p53 protein by E6 has a dual effect; it removes a proliferative brake from the cell and also leads to genomic instability and mutational inheritance (Saunders and Frazer, 1998). The E7 protein acts as a tumor-promoting agent by binding the tumor-suppressor genes pRb and p107. These tumor suppressors are negative regulators of the viral expression and proliferation-regulating factors, the E2 family. Thus, binding and inactivation of pRb, or p107 leads to the release of cells from negative growth signals and leads to unregulated growth control in the keratinocytes (Saunders and Frazer, 1998; Flaitz and Hicks, 1998). In instances of HPV-infection site transformation to malignancy, the viral DNA is integrated into the host DNA sequence. This integration often causes previously mentioned E2 gene (which regulates expression of viral genes and viral replication) to be disrupted (Hedge and Androphy, 1998), allowing the E5, E6, and E7 gene products to be produced in a completely deregulated fashion that leads to continued expansion of this malignant cell population. With the cellular controls by p53, 21, and p107 inactivated there is no mechanism to stop the uncontrolled differentiation.

Mosquito saliva of the mosquito Aedes aegypti has been found to contain two peptides:

Sialokinin I Asn-Thr-Gly-Asp-Lys-Phe-Tyr-Gly-Leu-Met-NH2

Sialokinin II Asp-Thr-Gly-Asp-Lys-Phe-Tyr-Gly-Leu-Met-NH2

(Champagne and Ribeiro, 1994)

They have been identified as being members of the tachykinin peptide family because they contain the carboxyl-terminal sequence Phe-X-Gly-Leu-Met-NH2 which is characteristic of this peptide family and is responsible for binding the tachykinin-specific receptors (Champagne and Ribeiro, 1994). These tachykinins, sialokinin I and sialokinin II (SK1 &SK2), have properties similar to tachykinin A and tachykinin B (TKA & TKB) as well as tachykinin substance P (SP) and neurokinin 1 NK1) (Champagne and Ribeiro, 1994). Sialokinin I and II (have been proposed to, because of their similarity to TKA, TKB, NK1, and SP) cause a number of physiologic and immune system changes in the mammalian body (Champagne and Ribeiro, 1994). Champagne and Ribeiro (1994) proposed the identity of the two mosquito peptides and proposed their enhancing behavior on mammal neutrophil phagocytosis and macrophage activation, but did not propose an application for these peptides in the cure of unregulated cellular proliferation.

The physiological and immune system changes in mammals caused by tachykinins include vasodilation, vascular permeability, activation of macrophages, activation of neutrophil granulocytes, T-lymphocyte proliferation, monocyte interleukin production, mast cell degranulation in epithelia, and eosinophil granulocytes degranulation (Lundberg, 1995). Although these characteristics are known of tachykinins, Lundberg has not proposed clinical uses of the mosquito tachykinins, such as sialokinin I and II.

When administered topically or by injection, the tachykinin family can be as effective as when released by their respective production sites (Champagne and Ribeiro, 1994; Lundberg 1995; Reeid et al 1993; Noveral and Grunstein, 1995). These statements are made about tachykinins in general by the above mentioned sources, but the implications of these functions shared by mosquito salivary tachykinins in their treatment of unregulated cellular proliferation were not recognized.

Tachykinins produce a variety of effects in addition to smooth muscle contraction and endothelium-dependent dilation. Substance P causes the release of histamine from mast cells and enhances human neutrophil phagocytosis, but these effects are dependent on the sequence of basis of amino acids (Arg-Pro-Lys-Pro) at the aminoterminal end and require micromolar concentrations in vitro or 100 nM concentrations in vivo. Some tachykinins that lack this sequence (possibly including the sialokinins) can inhibit the effect, because the carboxyl-terminal sequence is also involved in binding to a receptor on mast cells. On the other hand, substance P and neurokinins can activate macrophages at low concentrations, concentrations comparable to those required for smooth muscle contraction. As this activity is dependent on the carboxyl-terminal sequence, the sialokinins may also activate macrophages at the site of feeding. (Champagne and Ribeiro, 1994) Champagne and Ribeiro did not suggest (nor did their research make indications that would lead to suggesting) the use of these peptides to treat asthma or unregulated areas of cellular proliferation because so little is known as to the result of exposing damaged and irritated epithelia to these peptides. (There is in fact a danger that the inflammatory response of the body to these peptides will be severe.)

Cells lining the mosquito larval gut as well as the saliva gland of the water strider have been observed to display endopolyploidy because something appears to inhibit the cell from dividing after its chromosomes have multiplied (Klug and Cummings, 2000). This observation has been made, but no suggestion as to why has been proposed. This endopolyploidy is probably because of the action of tachykinins present in the gut of mosquitoes and very probably also in the salivary glands of water striders.

Studies of mammalian tachykinin NK1 receptor antagonist binding have shown that binding and blocking of certain tachykinin receptors by selective receptor antagonists can have an anti-tumor inhibiting activity in the mammalian body (Palma et al., 2000 and U.S. Pat. No. 5,990,125). Simply slowing or inhibiting a tumor is not the solution of the tumor's threat; elimination of the tumor removes the threat (as attested by the number of tumors which surgeon remove or destroy by radiation therapy every year), and thus the tackykinin NK1 receptor antagonist treatment does not go far enough. The critical step forward from a tumor inhibitor to tumor-fighting immune response stimulant was not recognized by the previous sources.

### SUMMARY OF THE INVENTION

Administering mosquito salivary tachykinins, such as sialokinin I and II, topically or by injection to areas of unregulated cellular proliferation “usually referred to as tumors, warts, sclerotic plaquing, fibrosis, focal lesions, or sarcoma” in the mammalian body will be successful in remediation of the problem area. “Unregulated cellular proliferation” as discussed in the scope of this proposal is undesirable cellular proliferation and differentiation as induced in the HPV example, but not exclusive to HPV tumors and warts alone. “Remediation of the problem area” as defined in this proposal, is the body's recovery of physiological and genetic control, disappearance of the unregulated cellular growth, and/or elimination of cancer danger in this tissue.

### BRIEF DESCRIPTION OF DRAWINGS

FIG. 1. (A simple model of the HPV infection mechanisms.)

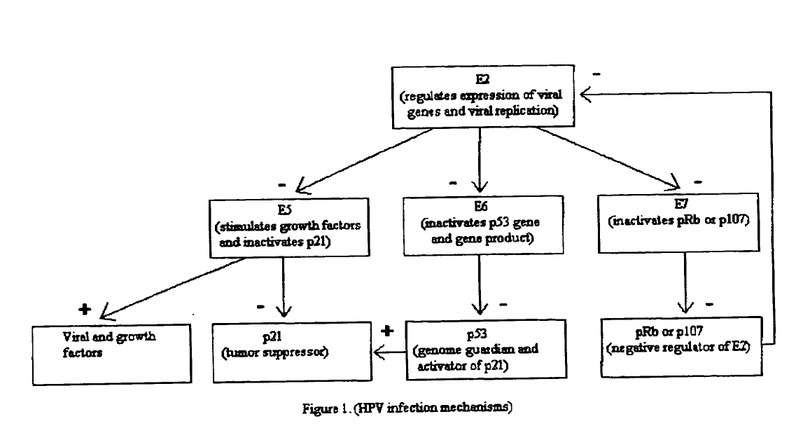

### DETAILED DESCRIPTION OF THE INVENTION

It is proposed that if a therapeutically effective amount of mosquito salivary tachykinins were injected into (similar to a mosquito bite and subsequent injection of its saliva), or applied topically to, unregulated mammalian tumor or wart tissue, that the previously mentioned immuno-physiologic mechanisms which they trigger would be induced. It is proposed that mosquito salivary tachykinins themselves inhibit cellular division by acting on the NK-receptors and that the immuno-physiologic mechanisms which they trigger also would cause the mammalian body to combat the unregulated cellular proliferation. An inflammatory response in the area of injection is expected. This inflammatory response should include activation of alveolar macrophages, neutrophil granulocytes, fibroblast proliferation, T-lymphocyte proliferation, monocyte interleukin production, and eosinophil granulocyte degranulation. Vasodilation, increased vascular permeability in the area of injection, and inhibition of mast cell degranulation is also expected. It is proposed that with these results a disappearance of the unregulated tissue by apoptosis of the cells in the tumor or wart. In cases of HPV, stimulation of the host organism's immune system to attack viral protein E6, allows mammalian p53 and p21 to reassume control and the polyp will destroy itself by self-induced cell death. It is proposed that the regeneration of fibroblasts and properly regulated epithelial proliferation necessary for repair of the area disrupted by unregulated cellular proliferation are also expected. It is proposed that as these cell growth regulatory processes transpire, disappearance of visible keratosis and wart structure, and elimination of cancer danger will be observed. In the HPV example, host immune inactivation of viral E5 protein will allow tumor suppressor gene p21 to function and regulate growth factor activity, controlling cell proliferation and stopping possible transformation to malignancy. Host immune attack on HPV E7 protein expression will enable the affected cells to resume tumor suppression through actions of pRb and p107.

The observed result of these claims will be the expected disappearance of the unregulated tissue structure, or tumor. Once the unregulated proliferation has been suppressed, mosquito salivary tachykinins also regulate epithelial proliferation in areas of epithelial damage inducing healing of the tumor-damaged area. Since removal of the tumor's threat is the most desired outcome, mosquito salivary tachykinins offer a minimally intrusive and nearly painless treatment to the menace. The use of invertebrate mosquito salivary tachykinins instead of a receptor antagonist in mammals will function to bind to the mammalian tachykinin receptor cites because of their chemical similarity to mammalian tachykinins TKA, TKB, SP, and NK1 and carry with them the powerful physiological and immune system stimulating abilities of the mosquito tachykinin on the mammalian body. Thus the use of mosquito salivary tachykinins on mammalian tumors will actually stimulate the mammalian body to destroy the tumor rather than simply inhibit its growth as in the case of the previously mentioned NK1 receptor antagonist.

It is claimed that:

1. Proteins of the tachykinin family in mosquito saliva, including sialokinin I and II, but not exclusively limited to sialokinin I and II, will inhibit cellular division and induce cells of vertebrate tissues to regain proliferative control in areas of unregulated cellular proliferation.

2. Proteins of the tachykinin family in mosquito saliva will also induce repair of the affected area of the cellular proliferation by immune response and apoptosis of the undesirable growth, but not exclusively limited to these mechanisms.

3. Mosquito salivary tachykinins, such as sialokinin I and II, are therapeutically effective when used singularly or in unison.

4. When mosquito salivary tachykinins, such as sialokinin I and II, are administered topically, by subcutaneous injection, or by intravenous injection (but not exclusively such methods), to regions of unregulated cellular proliferation they will retain their effectiveness as claimed above.

5. Mosquito salivary tachykinins are effective in the mammalian body because of their similarity to mammalian tachykinins which allows them to bind to the mammalian tachykinin receptor and, instead of acting as a tachykinin receptor antagonist, use this advantageous location to perform their immune system and regulatory control stimulatory functions.
